(19) United States
(12) Patent Application Publication
(10) Pub. No.: US 2021/0387794 A1
(43) Pub. Date: Dec. 16, 2021

(54) Medical Facemask Dispenser

(71) Applicant: Wisconsin Alumni Research Foundation, Madison, WI (US)

(72) Inventors: Joshua Glazer, Madison, WI (US); Adam McNeilly, Madison, WI (US)

(21) Appl. No.: 17/328,319

(22) Filed: May 24, 2021

Related U.S. Application Data
(60) Provisional application No. 63/038,332, filed on Jun. 12, 2020.

(51) Int. Cl. B65D 83/08; A61B 50/30; A61L 2/10

(52) U.S. Cl. CPC B65D 83/0829; A61B 50/30; B65D 83/0817; A61L 2202/26; A61B 2050/3013; B65D 2583/082; A61L 2202/122; A61L 2/10

(57) ABSTRACT

An automatic mask dispenser handles procedure masks of a type having filtration material inside ear loops by use of a high engagement material on a movable surface that can selectively remove the lowermost mask. In one embodiment opposed moving surfaces are provided to fold the mask hygienically about its inner surface for dispensing to the user.

### CROSS REFERENCE TO RELATED APPLICATION

This application claims benefit of U.S. provisional application 63/038,332 filed Jun. 12, 2020.

### STATEMENT REGARDING FEDERALLY SPONSORED RESEARCH OR DEVELOPMENT

NA

### BACKGROUND OF THE INVENTION

The present invention relates to automated dispensers for facemasks and in particular to a dispenser providing improved hygienic dispensing of stacked masks available from a variety of manufacturers.

Single-use disposable face masks, often termed “procedure masks,” and henceforth referred to simply as “masks,” are a form of personal protective equipment (PPE) widely used to reduce transmission of respiratory pathogens.

Such masks normally provide a pleated fabric panel providing filtration material formed of one or more internal filter layers sandwiched between outer layers providing improved comfort and abrasion resistance. Elastic ear loops may be attached on opposite sides of the filtration material to attach the mask over the mouth and nose. A malleable noseband may be affixed to an outer upper edge of the filtration material to improve the sealing of the mask around the user's nose when properly positioned.

Quantities of such procedure masks may be efficiently transported and stored in a stacked configuration with the fabric panels of filtration material fitting closely together and the ear loops loosely arrayed to the sides. Dispensing of the masks from the stack is normally accomplished through an open top or a slot in a carton holding the masks, for example, by reaching into this carton.

Recent CDC guidance encourages use of masks for infection control both in medical facilities and in public settings where proper social distancing for infection control may not be possible (e.g., grocery stores, pharmacies). Accordingly, it can be desirable to make such masks readily available in hospitals, clinics, and other venues to patients, employees, and the lay public.

Particularly during pandemics (e.g., COVID-19) when a huge demand for masks can lead to shortages, it is important to guard against hoarding and wasteful overuse of the masks. For this reason, the distribution of masks, for example, in a medical facility, is normally supervised by an individual who can monitor mask usage and ensure that masks are distributed in a hygienic state. This approach practically limits the number of locations where masks can be obtained, reducing their availability and incurring substantial costs.

### SUMMARY OF THE INVENTION

The present invention provides an automatic dispensing machine for procedure masks having a design that can effectively handle masks from a variety of manufacturers delivered in a stacked form. The automatic dispenser permits better allocation of masks to individuals with less waste and can guard against cross-contamination between dispensed and un-dispensed masks.

More specifically, one embodiment of the invention is an apparatus for dispensing masks of the type having filtration material for providing protection against pathogens, the filtration material affixed to side-extending ear loops. The apparatus provides a hopper for holding a stack of multiple masks with filtration material arranged in adjacent stacked layers and ear loops extending on left and right sides of the filtration material of each layer. A hopper bottom has both a slot opening for exposing the filtration material of a lowermost mask and surfaces outside of the slot resisting downward movement of the stack. A compressor biases the stack toward the hopper bottom and a motor driven extractor surface which is positioned to contact the filtration material of the lowermost mask when the stack is resting against the hopper bottom to urge the lowermost mask through the slot opening with motion of the extractor surface across the lower surface.

It is thus a feature of at least one embodiment of the invention to provide a mask dispenser that can manage stacks of masks reliably to reduce contamination and waste. It is another feature of at least one embodiment of the invention to provide a dispensing method that can accommodate masks with ear loops.

In one embodiment, the apparatus may include two transversely opposed motor driven extractor surfaces positioned to contact the filtration material of the lowermost mask when the stack is resting against the hopper bottom to urge opposite edges of the lowermost mask together to fold the mask as it moves through the slot.

It is thus a feature of at least one embodiment of the invention to provide a folding of the mask such as, for example, protects the mouth- and nose-facing side of the mask against contamination as it is being dispensed and removed from the dispenser.

The two transversely opposed motor driven extractor surfaces may provide opposing motion in contact with the filtration material of the lower most mask in the stack when that mask is in the stack and parallel motion away from the stack in contact with the folded mask as it is dispensed.

It is thus a feature of at least one embodiment of the invention to provide a dispenser mechanism that can not only remove a mask from a stack of masks but that can transport the mask away from the stack to a remote location reducing the possibility of cross-contamination from the user receiving the mask.

The apparatus may include a housing surrounding the hopper and the motor driven extractor surfaces and adapted to shield the hopper and motor driven extractor surfaces from external contamination, and the housing may include a dispenser slot positioned to allow dispensing of the mask in the folded state through the dispenser slot with the parallel motion of the mask away from the stack in contact with the motor driven extractor surfaces.

It is thus a feature of at least one embodiment of the invention to provide a dispensing apparatus that shields the masks from outside contamination.

The apparatus may include a controller controlling the motor driven extractor surfaces to stop motion of the mask after it has passed through the dispenser slot but before it is released from the motor driven extractor surfaces to allow final removal by a user.

It is thus a feature of at least one embodiment of the invention to prevent the need for the mask to touch an exterior surface that might be contaminated by suspending the mask to allow direct removal by the user.

The apparatus may include a sensor operatively communicating with the controller to stop the motor driven extractor surface when the sensor detects a position of the mask after it has passed through the dispenser slot but before it is released from the motor driven extractor surfaces.

It is thus a feature of at least one embodiment of the invention to allow precise exposure of the mask from the housing resistant to slippage or tolerance variations in the transport mechanism moving the mask to the slot.

The motor driven extractor surfaces may be belts passing around pulleys to present substantially collinear horizontal surfaces engaging filtration material of the lowermost mask to move the mask through the gap and to present substantially parallel spaced apart vertical surface belts conveying the masks downward to the dispenser slot.

It is thus a feature of at least one embodiment of the invention to provide a simple and robust mechanism that can both remove masks from a stack and transport them away from the stack.

The extractor surface may include a high engagement portion adapted to draw filtration material with it to urge the lowermost mask through the slot and a low engagement portion movable across a lowermost mask surface and adapted to slide without drawing filtration material along with it. Motion of the extractor surface may remove the high engagement portion from the stack as the lowermost mask is dispensed to prevent engagement with a mask above the lowermost mask in the stack as the lowermost mask is dispensed.

It is thus a feature of at least one embodiment of the invention to provide for the removal of masks using an engaging extractor surface while minimizing the possibility that multiple masks will be removed.

At a beginning of the extraction process, the high engagement surface may be positioned to not extend outside of transverse edges of the filtration material so that during dispensing of the mask the high engagement material does not contact a mask above the lowermost mask.

It is thus a feature of at least one embodiment of the invention to position the high engagement surface to always be separated by a mask being removed from the remaining masks of the stack to resist dispensing of multiple masks at one time.

The high engagement surface may provide an engagement directionality to produce higher engagement when the high engagement surface moves in a first direction with respect to the filtration material and a lower engagement when the high engagement surface moves in the opposite direction with respect filtration material.

It is thus a feature of at least one embodiment of the invention to provide a strong engagement between the engagement surface and the filter material to disengage the filter material from the stack while allowing a weak engagement between the engagement surface in the filter material when the user pulls on the partially dispensed mask.

The high engagement surface may be a set of angled barbs.

It is thus a feature of at least one embodiment of the invention to provide a high engagement surface that exhibits directionality using features that provide good engagement with a fibrous mask material.

The apparatus may include an authorization interface for receiving a user identification, the authorization interface communicating with a controller controlling operation of the motor driven extractor surfaces and operating to record the user identification identifying a user

It is thus a feature of at least one embodiment of the invention to provide a method of monitoring mask usage thus reducing overuse.

The apparatus may include a wireless transmitter for transmitting badge information user identification to a remote location.

It is thus a feature of at least one embodiment of the invention to provide simplified central logging of mask usage.

The authorization interface may communicate with a controller and with a list of authorized users to prevent operation of the motor driven extractor surfaces when the user identification is not on the list of authorized users.

It is thus a feature of at least one embodiment of the invention to reduce mask diversion by unauthorized individuals.

The authorization interface may communicate with a controller controlling operation of the motor driven extractor surfaces and operates to limit the dispensing of masks to a given individual for a predetermined period of time after a previous dispensing of a mask to the given individual.

It is thus a feature of at least one embodiment of the invention to promote efficient mask reuse during a given interval of time.

The apparatus may include a sensor for communicating with the motor driven extractor surfaces to activate the motor driven extractor surfaces with detection of motion of a user hand or the like.

It is thus a feature of at least one embodiment of the invention to allow hands-free activation of the dispenser to reduce cross-contamination, for example, if a common mechanical button were required.

The apparatus may include sterilizing ultraviolet lights for sterilizing mask surfaces of the apparatus that may contact the masks.

It is thus a feature of at least one embodiment of the invention to reduce cross-contamination that might otherwise occur in a common PPE dispensing point.

The apparatus may include a fan for discharging filtered air into a housing adapted to shield the hopper and motor driven extractor surfaces from external contamination, the fan operating during activation of the motor driven extractor surfaces.

Is thus a feature of at least one embodiment of the invention to create a positive pressure within the housing to eject lint and debris and reduce cross-contamination from a user's hands positioned near the opening in the housing.

The hopper may further include vertically extending loop retention elements separating the ear loops from the filtration material in the hopper.

It is thus a feature of at least one embodiment of the invention to reduce any propensity of the loops associated with the masks from tangling or causing multiple masks to be dispensed at a single time

These particular objects and advantages may apply to only some embodiments falling within the claims and thus do not define the scope of the invention.

### BRIEF DESCRIPTION OF THE DRAWINGS

FIG. 1 is an exploded perspective view of a dispenser constructed according to one embodiment of the invention showing a hopper for receiving procedure masks and a belt system for extracting those masks and delivering them to a slot at a bottom of the dispenser housing;

FIG. 2 is a perspective view of the embodiment of FIG. 1 assembled within the housing to be mounted to a wall or the like;

FIGS. 3a-3d are simplified vertical elevational representations of the belts of FIG. 1 showing multiple stages of removing a mask from a stack of masks and delivering it for receipt by an individual and a resetting of the belts for subsequent dispensing;

FIG. 4 is a fragmentary perspective view of one belt of the belt drive system of FIG. 3 showing a localized high engagement surface;

FIG. 5 is a detailed fragmentary view of the high engagement surfaces of the belts of FIG. 3 showing the directionality of the engagement that allows extraction of the mask when the belts are still; and

FIG. 6 is a schematic representation of the principal electrical components and connections of the present invention.

### DETAILED DESCRIPTION OF THE PREFERRED EMBODIMENT

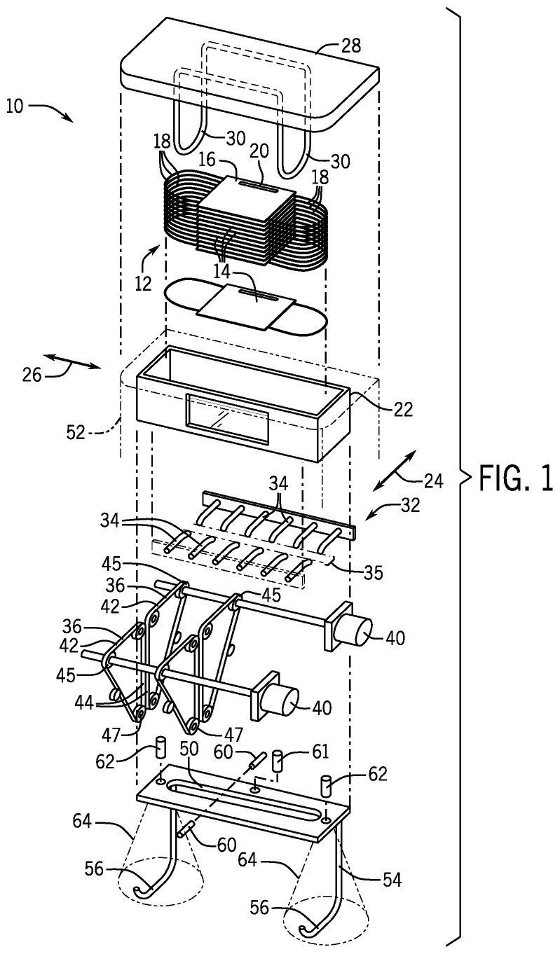

Referring now to FIG. 1, an automated mask dispenser system 10 may receive a stack 12 of masks 14, for example, the latter each providing for laminated fabric panel 16 sized to fit over a user's mouth and nose when drawn against the user's face by elastic ear loops 18 extending from left and right sides of the fabric panel 16. A malleable nose bridge 20 may be affixed to one edge of the outer surface of the fabric panel 16 to allow the mask 14 to better conform to the user's nose for improved sealing. Generally, the stack 12 may assemble the fabric panel 16 in closely adjacent and abutting layers for high volumetric density with the ear loops 18 spreading loosely outward from edges of the fabric panel 16. Masks 14 of this kind are commercially available from a wide variety of vendors and may make use of woven or nonwoven fabrics of synthetic or natural materials as is generally understood in the art.

The masks 14 may be received as a stack 12 in a generally rectangular hopper 22 open at the top and bottom and having a transverse width 24 generally matching the transverse width of the fabric panel 16 and a longitudinal width along longitudinal axis 26 sufficient to receive the fabric panel 16 and the ear loops 18 without compressing the ear loops 18 toward the fabric panel 16. In this latter regard, a lid 28 fitting against the upper surface of the hopper 22 may be provided having downwardly extending expander loops 30 that may be received within the ear loops 18 to spread elastic of the ear loops 18 apart and to separate the ear loops 18 from the fabric panel 16 to reduce tangling and ensure that the ear loops 18 are not folded back into the stack of fabric panel 16 during dispensing. It will be appreciated that the hopper 22 may be amply sized to handle many more masks than are typically shipped in a container so that multiple stacks 12 can be stacked on top of each other. Other methods of segregating the ear loops 18 may also be employed including, for example, a vertical constricting channel at either longitudinal edge of the hopper 22.

The stack 12, when received within the hopper 22, may rest against a hopper bottom 32, for example, comprised of inwardly extending horizontal opposed arrays of cantilevered prongs 34 against which the lower surface of a lowermost mask 14 of the stack 12 may rest when the stack 12 is received within the hopper 22. The prongs 34 expose the lower surface of the fabric panel of the lowermost mask 14 between the prongs 34. The opposed cantilevered tips of the prongs 34 are separated to define a transverse gap 35 therebetween extending along the longitudinal direction.

As depicted, and for clarity in showing the nose bridge 20, the inner surface of the masks 14, as worn, are oriented downwardly; however, preferably the outer surface of the masks 14 away from the user's mouth and nose during use, will be downwardly facing to provide improved hygienic handling of the masks 14 upon dispensing as will be discussed below.

Positioned between prongs 34 to also contact the lower surface of the lowermost masks 14 in the received stack 12 are a set of elastomeric belts 36. These elastomeric belts 36 may be driven in the transverse direction, as will be discussed below, to provide an extraction surface that can engage the material of the fabric panel 16 of the mask 14 to pull the lowermost mask 14 through the gap 35. Generally, there may be four elastomeric belts 36 arranged in pairs, each pair providing two elastomeric belts 36 in transverse opposition across the gap 35 and one pair separated from the other pair longitudinally about a center of the fabric panel 16 as aligned on the prongs 34. The belts 36 on different sides of the gap 35 may, for example, be timing belts having internal teeth received by corresponding teeth in pulleys 45 that are driven separately by independently controlled stepper motors 40 as will be discussed below.

Other un-driven or idler pulleys 47 may guide a remainder of a loop of each belt to define multiple outer belt surfaces. Importantly, each belt 36 in a dispensing position, depicted in FIG. 1, may provide a substantially horizontal belt upper surface 42 that may contact the lower portion of the mask 14 through the prongs 34. This horizontal belt upper surface 42 transitions to a vertical surface 44 centered at and extending away from the gap 35.

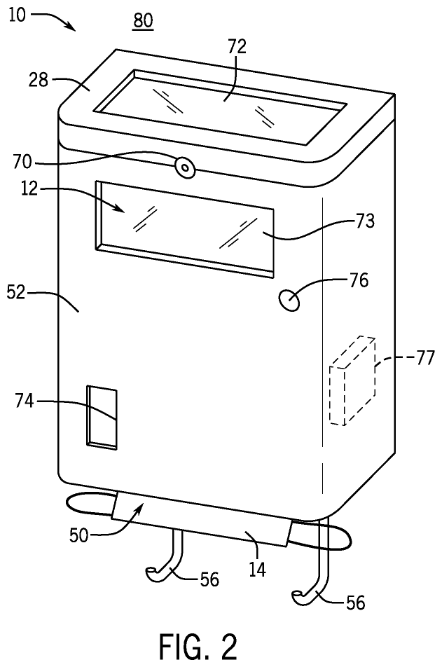

Referring also to FIG. 2, when the belts are in the dispensing position as shown in FIG. 1, the horizontal belt upper surface is 42 of each pair will generally move in opposite directions toward the gap 35 to pull the lowermost mask 14 downward into the opposed belt vertical surfaces 44 of that belt pair so that the mask is moved to and dispensed through a lower dispenser slot 50 generally vertically aligned with the gap 35. This dispenser slot 50 provides a transition from an interior of a housing 52 holding the hopper 22, the prongs 34, the belts 36, and their drive system, to an exterior outside of the dispenser system 10 where the mask 14 can be retrieved by a user. Positioned beneath the dispenser slot 50 may be an optional catcher frame 54 having a downwardly extending wire form terminating in a horizontally extending hook 56 positioned to catch the mask 14 if it falls from the dispenser slot 50.

Positioned along the path of the masks 14 as they pass downward, held between the belt vertical surfaces 44 of the belts 36, is an emitter/detector sensor pair 60 which can confirm positioning of the mask 14 ideally with a portion extending out of the slot 50 and a portion still retained in part between the belt vertical surfaces 44 of the belt 36. Ultraviolet sterilizing lamps 62 may be positioned with respect to the slot 50 to provide a sterilizing light field 64 downward that may be used to clean the mask-contacting surfaces of the catch hooks 56 and/or exterior surfaces of the slot 50.

A proximity detector 61, for example, using a passive infrared (PIR) detector, and infrared emitter detector pair arranged as a gesture or proximity sensor generally understood in the art, may be positioned to detect a hand motion or presence beneath the slot 50 to activate the dispenser system 10 as will be discussed below.

Referring still to FIG. 2, the lid 28 may fit tightly to a remainder of the housing 52 to provide an enclosed volume protecting the stack 12 therein. A locking mechanism 70 may be used to attach the lid 28 to the remainder of the housing 52 to prevent unauthorized removal or contamination of the stack 12. The locking mechanism 70 may be associated with an electrical switch to indicate that the lid 28 has been removed, for example, as may indicate a refilling of the dispenser system 10. Sealed windows 72 and 73 may be provided in the lid 28 and in a front surface of the housing 52 to allow visual inspection of the stack 12 to determine whether additional masks 14 need to be loaded. This need for additional masks may also be determined by counting the number of dispensings that occur in between each loading indicated by removal of the lid 28 or by a separate sensor system.

A front of the housing 52 may also expose an authorization interface 74 allowing entry of a user identification number by a user of the dispenser system 10, for example, by badge scanning using any of a magnetic stripe reader, RFID tag reader, optical reader, a keypad or the like. A service light 76 may be also be exposed at the front of the housing 52 to allow a simple indication of the need for service of the dispenser system 10, for example, in the event of a mask jam or lack of masks 14. The housing 52 may provide an inlet in one wall communicating with a contained filter and fan 77 that serves to provide filtered air to the interior of the cabinet to help discharge dust and lint from the masks that might otherwise prematurely clog the dispenser mechanism and to maintain a positive pressure during dispensing reducing potential contamination.

The housing 52 may be attached to a wall 80, for example, using tamper-resistant fasteners or other techniques to prevent ready removal without access to the interior of the housing 52, that requires a key for the locking mechanism 70.

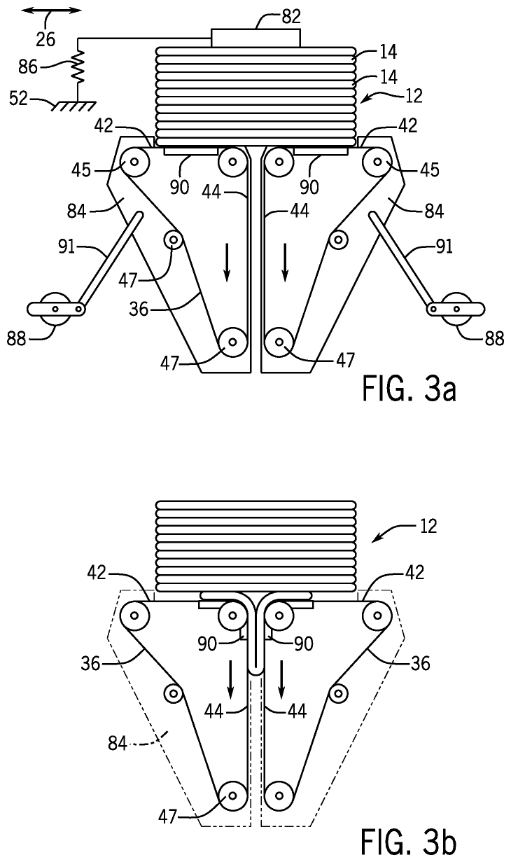

Referring now to FIGS. 1 and 3a, prior to activation of the dispenser system 10 to dispense a mask 14, pulley carriers 84 holding multiple pulleys 45 and 47 may be in a first engaged position as depicted pivoting together about pivot points aligned with pulleys 45 so that the upper surfaces 42 of the belts 36 are substantially horizontal and the belt vertical surfaces 44 are in substantially vertical opposition. This positioning may be performed by servomotors 88 connected to the pulley carriers 84 by linkages 91.

At this time, an installed stack 12 of masks 14 may be pressed against the belt upper surfaces 42 of the belts 36 to be supported both by the belt upper surfaces 42 and the prongs 34 (not visible in FIG. 3a). This downward biasing of the stack 12 may be, for example, by means of a weight or a cantilevered arm pressing on the top of the stack 12 and biased downward with respect to the housing 52 by a linear spring 86 or the like.

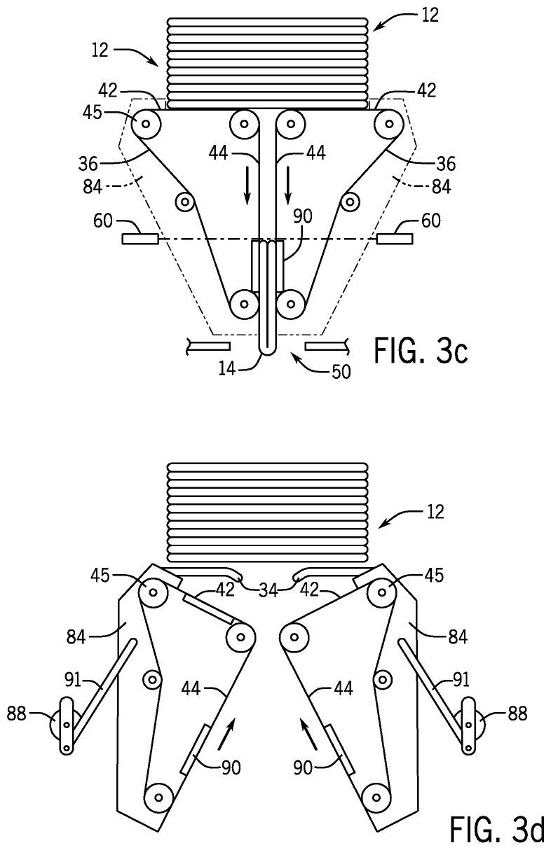

Referring now to FIG. 4, in positioning of the pulley carriers 84 prior to dispensing of a mask 14, a high engagement surface 90 forming a portion of the outer surface of each belt 36 may be aligned at the transverse outer edges of the stack 12 to extend arbitrarily toward the center of the stack 12 but typically by the remainder of the length of the belt upper surface 42. This high engagement surface 90, for example, may provide for a set of barbs or hooks 92 directed inwardly along a plane of the belt upper surface 42 toward the gap 35 to promote engagement between the belt 36 and fibrous material forming the outer surfaces of the fabric panels 16 of the masks 14 in the stack 12. In one example, the barbs or hooks 92 may be similar to those used in hook and loop fasteners such as Velcro™. Ideally the barbs or hooks 92 provide a directional engagement tending to engage the fabric panels 16 when the relative motion of the high engagement surface 90 with respect to the masks 14 is toward the center of the fabric panels 16 (as depicted) and to disengage from the material of the fabric panels 16 when the relative motion is in the opposite direction with counter rotation of the pulley 45. The invention contemplates a wide variety of high engagement surfaces 90 may be used relying not simply on barbs but alternatively or in addition a stickiness of the high engagement surface 90 compared to the remainder of the belt 36 or greater surface roughness as well as features or protrusions that serve to selectively engage the mask fabric panels 16.

The remainder of the belt 36 outside of the high engagement surfaces 90 will be relatively smooth to provide minimal or no engagement with the fabric panels 16 as it moves with respect to the masks 14. Generally, the region outside of the high engagement surfaces 90 will not sufficiently engage the fabric panel 16 to urge the fabric panel 16 through the gap 35 and will have less engagement force with the fabric panel 16 than the high engagement surface 90 in at least one direction by a factor of at least two. The region outside of the high engagement surfaces 90 may, for example, be smooth or smoother than the high engagement surfaces 90 and/or may have an absence of barbs or hooks 92 or barbs or hooks 92 facing in a different direction.

Referring now to FIGS. 1 and 3b, activation of the dispenser system 10 to dispense a mask 14, for example, as triggered by the proximity detector 61 described with respect to FIG. 1, or swiping or reading of a badge through authorization interface 74 shown in FIG. 2 may cause rotation of the motors 40 so as to move the upper surfaces 42 of the transversely opposed belts 36 inward gripping the lower surface of the fabric panel 16 of the lowermost mask 14 and causing this lowermost mask 14 to fold in half along the longitudinal axis 26 as it passes downwardly through the space between the belt vertical surfaces 44 of adjacent moving belts 36. As the high engagement surfaces 90 move inwardly, the high engagement surfaces 90 move away from contact with a lower surface of the next higher mask 14 in the stack 12. This next higher mask 14 is exposed only to regions of the belts 36 outside of the high engagement surfaces 90 which are insufficient to induce the dispensing of the next higher mask 14. The process of folding the mask 14 requires a force of engagement with the fabric panel 16 that can only be provided by the high engagement surface 90 and cannot be provided by either contact with other areas of the belt 36 or the friction between adjacent masks 14 thus reducing any chance of multiple mask dispensing at a single time.

Referring now to FIGS. 1 and 3c, during a dispensing process as triggered, motion of the belts 36 continues for predetermined number of motor steps (when stepping motors 40 are used) or until there is a breaking and reestablishing of a light beam between the sensor pair 60 indicating that the mask 14, as folded and held between the belt vertical surfaces 44, protrudes by a predetermined amount through the slot 50 to be accessible by the user desiring a mask 14.

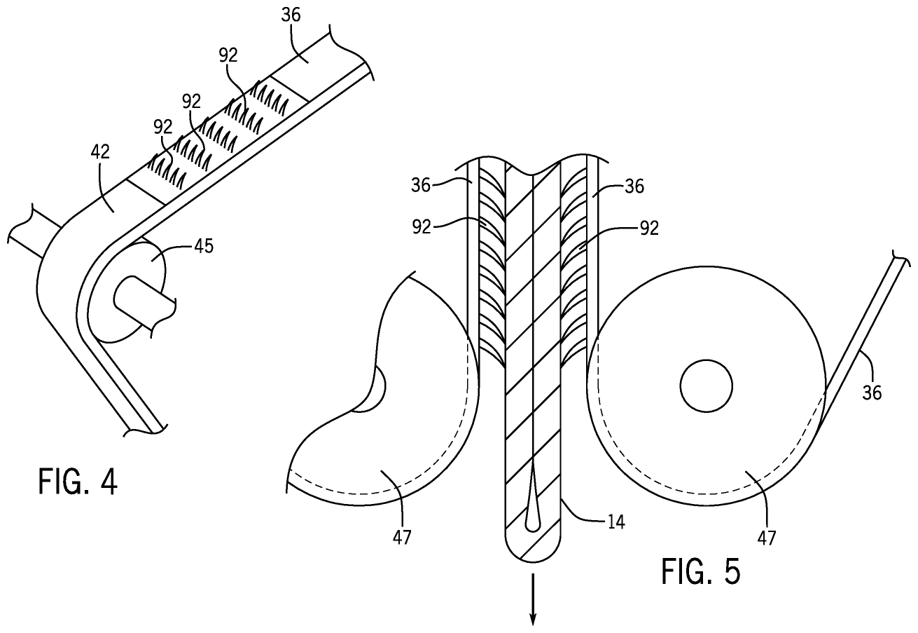

Referring also to FIG. 5, when the mask 14 is so positioned, motion of the belts 36 ceases and the directionality of the engagement of the high engagement surfaces 90 allows the user to grasp and pull downward on the mask 14 to draw it fully out of the dispenser system 10 through the slot 50 without reduced resistance from the high engagement surface 90. This dispensing process which suspends the mask 14 allows controlled dispensing without the need for the mask to fall on hooks 56 or be exposed to other contamination. Removal of the mask 14 may be detected, for example, by a separate sensor (not shown) or may be assumed after a given lapse of time.

After the mask 14 has been removed, the motors 40 may be reactivated to fully discharge any residual material on the belts from the housing 52. At all times during the dispensing and up until removal of the mask, the fan 77 may be activated to provide a downdraft of filtered air to reduce contamination of the interior of the housing 52.

Referring now to FIGS. 1 and 3d, after this clearing process, pulley carriers 84 under the control of the servomotors 88 swing away from each other about pivot points centered on the pulleys 45 with the belt upper surfaces 42 dropping downward and the vertical surfaces 44 tipping away from each other and outward. At this time, the belts 36 are operated in the reverse direction by control of the motors 40 moving the high engagement surfaces 90 back to their position shown in FIG. 3a while removed from contact with the mask stack 12. The amount of necessary motion may be determined by timing, step counting (for stepper motors), the use of mechanical stops or sensors, or other techniques well known in the art. When the high engagement surfaces 90 are restored to their previous position of FIG. 3a, the servomotors 88 are reactivated to move the pulley carriers 84 back to the position shown in FIG. 3a.

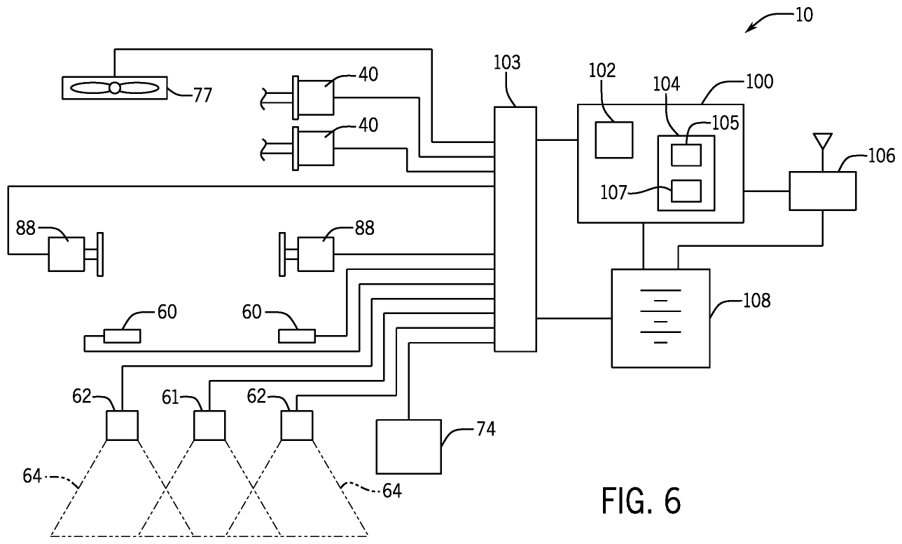

Referring now to FIG. 6, the above described dispenser system 10 may be controlled by a controller 100 having one or more processors 102 communicating with electronic memory 104 holding a stored program 105 to implement the steps described above. The memory 104 may also hold a list of authorized users 107 to compare against user identification provided through the authorization interface 74 and thereby to determine whether the user is authorized to receive a mask 14. The memory 104 may be also be used to log each dispensing occurrence together with the associated user's identification. In this respect, the controller 100 may communicate each dispensing occurrence and the associated user through the wireless transceiver 106 to a central location where this information may be collected or monitored. The stored program 105 may also operate to limit the number of masks dispensed during a given interval of time determined by a processor clock or the like.

The controller 100 may connect with an interface 103 providing high power solid-state or relay switching elements to provide control signals to the various other components of the dispenser system 10 and isolation circuits for receiving signals from received from various other sensors described above with respect to the dispenser system 10. More specifically, the interface 103 may provide control signals to the motors 40 with respect to direction and number of steps, the fan 77, the servomotors 88, and the ultraviolet sterilizing lamps 62. Likewise the interface 103 may receive signals from the sensor pair 60, the proximity detector 61 and the authorization interface 74. As noted, the controller 100 may also communicate with a wireless transceiver, for example, a Wi-Fi transceiver 106 which may be used, for example, to control authorization to use the dispenser system 10 according to badge identification and record usage by individuals having different badge numbers. This transceiver 106 may also transmit indication of any jamming of the mechanism of the dispenser system 10 or an exhaustion of masks 14 to allow reloading of the hopper 22. All of the systems described above may be powered by an internal battery 108 and/or a line voltage supply suitably processed with respect to voltage.

The term “fabric” as used herein refers to both woven and nonwoven fabrics and similar flexible materials having porous surfaces suitable for filtration of a user's breath.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference, which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

References to “a microprocessor” and “a processor” or “the microprocessor” and “the processor,” can be understood to include one or more microprocessors that can communicate in a stand-alone and/or a distributed environment(s), and can thus be configured to communicate via wired or wireless communications with other processors, where such one or more processor can be configured to operate on one or more processor-controlled devices that can be similar or different devices. Furthermore, references to memory, unless otherwise specified, can include one or more processor-readable and accessible memory elements and/or components that can be internal to the processor-controlled device, external to the processor-controlled device, and can be accessed via a wired or wireless network.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

What we claim is:

1. An apparatus for dispensing masks of a type having filtration material for providing protection against pathogens, the filtration material affixed to side extending ear loops, the apparatus comprising:
a hopper for holding a stack of multiple masks with filtration material arranged in adjacent stacked layers and ear loops extending on left and right sides of the filtration material of each layer;
a hopper bottom providing a slot opening for exposing the filtration material of a lowermost mask and surfaces outside of the slot resisting downward movement of the stack;
a compressor biasing the stack toward the hopper bottom; and
a motor driven extractor surface positioned to contact the filtration material of the lowermost mask when the stack is resting against the hopper bottom to urge the lowermost mask through the slot opening with motion of the extractor surface across the lower surface.

2. The apparatus of claim 1 providing two transversely opposed motor driven extractor surfaces positioned to contact the filtration material of the lowermost mask when the stack is resting against the hopper bottom to urge opposite edges of the lowermost mask together to fold the mask as it moves toward the slot.

3. The apparatus of claim 2 wherein the two transversely opposed motor driven extractor surfaces provide opposing motion in contact with the filtration material of the lowermost mask in the stack when that mask is in the stack and parallel motion away from the stack in contact with the folded mask as it is dispensed.

4. The apparatus of claim 3 further including a housing surrounding the hopper and the motor driven extractor surfaces and adapted to shield the hopper and motor driven extractor surfaces from external contamination, wherein the housing includes a dispenser slot positioned to allow dispensing of the mask in the folded state through the dispenser slot with the parallel motion extractor services moving the mask away from the stack.

5. The apparatus of claim 4 further including a controller controlling the motor driven extractor surfaces to stop motion of the mask after it has passed through the dispenser slot but before it is released from the motor driven extractor surfaces to allow final removal by a user.

6. The apparatus of claim 5 further including a sensor operatively communicating with a controller to stop the motor driven extractor surface when the sensor detects a position of the mask after it has passed through the dispenser slot but before it is released from the motor driven extractor surfaces.

7. The apparatus of claim 4 wherein the motor driven extractor surfaces are belts passing around pulleys to present substantially collinear horizontal surfaces engaging filtration material of the lowermost mask to move the mask through the slot opening and to present substantially parallel spaced apart vertical surface belts conveying the masks downward to the dispenser slot.

8. The apparatus of claim 1 wherein the extractor surface includes a high engagement portion adapted to draw filtration material with it to urge the lowermost mask through the slot and a low engagement portion movable across a lowermost mask surface and adapted to slide without drawing filtration material along with it;
and wherein motion of the extractor surface removes the high engagement portion from the stack as the lowermost mask is dispensed to prevent engagement with a mask above the lowermost mask in the stack as the lowermost mask is dispensed.

9. The apparatus of claim 8 wherein at a beginning of a mask dispensing, the high engagement surface does not extend outside of transverse edges of the filtration material so that during dispensing of the mask the high engagement material does not contact a mask above the lowermost mask.

10. The apparatus of claim 8 wherein the high engagement surface provides an engagement directionality to produce higher engagement when the high engagement surface moves in a first direction with respect to the filtration material and a lower engagement when the high engagement surface moves in an opposite direction with respect to filtration material.

11. The apparatus of claim 10 wherein the high engagement surface is a set of angled barbs.

12. The apparatus of claim 1 further including an authorization interface for receiving a user identification, the authorization interface communicating with a controller controlling operation of the motor driven extractor surfaces and operating to record the user identification identifying a user.

13. The apparatus of claim 12 further including a wireless transmitter for transmitting badge information user identification to a remote location.

14. The apparatus of claim 1 further including an authorization interface for receiving a user identification, the authorization interface communicating with a controller and with a list of authorized users to prevent operation of the motor driven extractor surfaces when the user identification is not on the list of authorized users.

15. The apparatus of claim 1 further including an authorization interface for receiving a user identification, the authorization interface communicating with a controller controlling operation of the motor driven extractor surfaces and operating to limit the dispensing of masks to a given individual for a predetermined period of time after a previous dispensing of a mask to the given individual.

16. The apparatus of claim 1 further including a sensor for communicating with the motor driven extractor surfaces to activate the motor driven extractor surfaces with detection of motion of a user hand or the like.

17. The apparatus of claim 1 further including sterilizing ultraviolet lights for sterilizing mask surfaces of the apparatus that may contact the masks.

18. The apparatus of claim 1 further including a fan for drawing and discharging filtered air into a housing adapted to shield the hopper and motor driven extractor surfaces from external contamination, the fan operating during activation of the motor driven extractor surfaces.

19. The apparatus of claim 1 wherein the hopper further includes vertically extending loop retention element separating the ear loops from the filtration material in the hopper.
